(19) United States
(12) Patent Application Publication
(10) Pub. No.: US 2022/0000359 A1
(43) Pub. Date: Jan. 6, 2022

(54) AUTOMATED REFRACTION EXAMINATION SYSTEM

(71) Applicant: Zaaf Science Group LLC, Newington, CT (US)

(72) Inventor: Yan Zhang, Newington, CT (US)

(21) Appl. No.: 17/364,258

(22) Filed: Jun. 30, 2021

Related U.S. Application Data
(60) Provisional application No. 63/046,715, filed on Jul. 1, 2020.
(60) Provisional application No. 63/107,392, filed on Oct. 29, 2020.

(51) Int. Cl. A61B 3/028; A61B 3/032; A61B 3/00; A61B 3/14

(52) U.S. Cl. CPC A61B 3/0285; A61B 3/032; A61B 3/14; A61B 3/0041; A61B 3/0008; A61B 3/0033

(57) ABSTRACT

A system for conducting an automated refraction examination on a patient, including: a phoropter configured to present different optics to the patient; an automated reading rod mounted to the phoropter; and a control unit configured to send signals to perform an automated refraction examination; wherein the phoropter comprises a right member with a right aperture and a left member with a left aperture; wherein the automated reading rod comprises: a reading rod including a first end and a second end; a motor coupled to the first end of the reading rod, wherein the motor is configured to drive the reading rod between a first position and a second position; a mounting interface configured to mount the automated reading rod to the phoropter; a reading card holder coupled to the second end of the reading rod; and a reading card coupled to the reading card holder.

### CROSS-REFERENCE TO RELATED PATENT APPLICATIONS

This application claims priorities from provisional application No. 63/046,715, filed Jul. 1, 2020, and from provisional application No. 63/107,392, filed Oct. 29, 2020, the content of which are incorporated herein in the entirety by references. Some references, which may include patents, patent applications and various publications, are cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference is individually incorporated by reference. In terms of notation, hereinafter, [n] represents the nth reference cited in the reference list. For example, [1] represents the first reference cited in the reference list, namely, GMSA, “GSMA intelligence,” https://www.gsmaintelligence.com/, Accessed On: February 2019.

### TECHNICAL FIELD

The present disclosure relates to the field of optometry, and more particularly relates to a phoropter for conducting an automated refraction examination.

### BACKGROUND

A refraction examination is typically used to produce an eyeglass prescription for a patient based on subjective responses given to a medical practitioner, such as an optometrist. While objective refraction techniques may be used to automatically determine a patient's eyeglass prescription, objective refraction techniques, such as when using an autorefractor, are typically inaccurate and are used as a starting point for a subjective refraction examination. A conventional subjective refraction examination requires interaction between a medical practitioner and a patient, as the patient's responses are required to determine optimal eyeglass prescription values. For example, when testing for a sphere value of the patient's right eye, two views using two optics of different sphere values may sequentially be presented to the patient for comparison. The medical practitioner typically adjusts a phoropter between the two optics such that the patient can compare the two views. Based on the patient's response (e.g. the patient may indicate that a first view is clearer than a second view), the spherical value for the patient's right eye may be adjusted. The eyeglass prescription values that may be adjusted typically include the sphere value, a cylinder value, an axis value, an add value, and a prism value. During a refraction examination, a near vision test may be conducted to determine the patient's visual acuity at a close range and relates to the patient's add value. Typical near vision tests display a series of letters at a short distance from the patient, and the medical practitioner may ask the patient to read the letters. Displaying the letters at an appropriate distance from the patient is essential to performing an accurate near vision test.

Conventionally, a manual reading rod may be used in combination with a phoropter to display the letters to the patient. When a near vision test is required, the manual reading rod (initially mounted in a vertical position on the phoropter) may be pushed down into a parallel position such that a reading card coupled to the manual reading rod is shown to the patient. However, the conventional, manual reading rod requires a medical practitioner, assistant, etc. to physically bring down and, when the near vision test is over, stow the rod away. Such a configuration requires that a medical practitioner, assistant, etc. is present in the room to conduct a near vision test.

Therefore, a heretofore unaddressed need exists in the art to address the aforementioned deficiencies and inadequacies.

### SUMMARY

In one embodiment, a phoropter for conducting an automated refraction examination on a patient is described. The phoropter includes: a right member with a right aperture; a left member with a left aperture; an upper member connecting to the right member and the left member; a mounting interface coupled to the upper member; a control unit configured to send signals to conduct the automated refraction examination; and an automated reading rod mounted to the phoropter via the mounting interface, the automated reading rod comprising: a reading rod comprising a first end and a second end; a motor coupled to the first end of the reading rod, wherein the motor is configured to drive the reading rod between a first position and a second position; a reading card holder coupled to the second end of the reading rod; and a reading card coupled to the reading card holder.

In another embodiment, the control unit is configured to communicate with the patient via an input device and an output device; wherein the output device comprises one or more of a speaker and a display; and wherein the input device comprises one or more of a microphone, a keyboard, a joystick, and a camera.

In another embodiment, the control unit communicates with the patient to adjust the phoropter during an automated refraction examination.

In another embodiment, the phoropter is configured to output results of the automated refraction examination to a database.

In another embodiment, the mounting interface includes: a mounting adapter coupled to the phoropter; a mounting block coupled to the mounting adapter; and a mounting bracket coupled to the mounting block; wherein the motor is coupled to the mounting bracket.

In another embodiment, the first position is an active position and the second position is an inactive position.

In another embodiment, the motor further includes a motor shaft and a cable is coupled to the motor shaft and to the second end of the reading rod such that actuating the motor drives the reading rod between the first position and the second position.

In another embodiment, the phoropter further includes a light source, wherein the light source is coupled to the reading rod between the reading card holder and the motor and is configured to illuminate the reading card.

In another embodiment, the control unit controls the light source to illuminate the reading card.

In another embodiment, the phoropter, further includes a camera module, wherein the camera module is coupled to the reading rod between the reading card holder and the motor and is configured to capture images of the patient's pupils.

In another embodiment, the camera module comprises one or more secondary light sources configured to illuminate one or more pupils of the patient.

In another embodiment, the control unit controls the camera module to capture images of the patient's pupils.

In another embodiment, the mounting adapter is coupled to a front surface of the upper member.

In another embodiment, the mounting adapter is coupled to the phoropter between the left member and the right member to a lower surface of the upper member.

In another embodiment, the reading card is an electronic display.

In another embodiment, the control unit controls the electronic display to display lines of letters, words or images to the patient.

In another embodiment, the controller is configured to communicate wirelessly with the motor.

In another embodiment, the controller communicates via a wired connection with the motor.

In another embodiment, the phoropter further includes a level indicating a balance status of the phoropter.

In another embodiment, the level is an accelerometer.

### BRIEF DESCRIPTION OF THE DRAWINGS

The accompanying drawings illustrate one or more embodiments of the present disclosure and, together with the written description, serve to explain the principles of the present disclosure, wherein:

FIG. 1 illustrates a block diagram of an exemplary system for conducting an automated refraction examination, in accordance with an embodiment of the present disclosure;

FIG. 2 illustrates an exemplary automated refraction system, in accordance with an embodiment of the present disclosure;

FIG. 3 illustrates an exemplary automated reading rod, in accordance with an embodiment of the present disclosure;

FIG. 4 illustrates an exemplary phoropter with a first mounting adapter, in accordance with an embodiment of the present disclosure;

FIG. 5 illustrates an exemplary phoropter with a second mounting adapter, in accordance with an embodiment of the present disclosure;

FIGS. 6A-6B illustrate an exemplary phoropter, wherein FIG. 6A shows a perspective view of a phoropter and FIG. 6B shows a front view of a phoropter, in accordance with an embodiment of the present disclosure;

FIG. 7 illustrates an exemplary camera module, in accordance with an embodiment of the present disclosure;

FIG. 8 illustrates an automated refraction cable system, in accordance with an embodiment of the present disclosure; and

FIG. 9 illustrates an exemplary optometry examination system, in accordance with an embodiment of the present disclosure.

### DETAILED DESCRIPTION

The present disclosure will now be described more fully hereinafter with reference to the accompanying drawings, in which exemplary embodiments of the present disclosure are shown. The present disclosure may, however, be embodied in many different forms and should not be construed as limited to the embodiments set forth herein. Rather, these embodiments are provided so that this disclosure is thorough and complete, and will fully convey the scope of the present disclosure to those skilled in the art. Like reference numerals refer to like elements throughout.

The terms used in this specification generally have their ordinary meanings in the art, within the context of the present disclosure, and in the specific context where each term is used. Certain terms that are used to describe the present disclosure are discussed below, or elsewhere in the specification, to provide additional guidance to the practitioner regarding the description of the present disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It is appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the present disclosure or of any exemplified term. Likewise, the present disclosure is not limited to various embodiments given in this specification.

It is understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It is understood that, although the terms Firstly, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present disclosure.

It is understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It is also appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the present disclosure. As used herein, the singular forms “a,” “an,” and “the” are intended to include the multiple forms as well, unless the context clearly indicates otherwise. It is further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It is understood that relative terms are intended to encompass different orientations of the device in addition to the orientation shown in the figures. For example, if the device in one of the figures is turned over, elements described as being on the “lower” side of other elements will then be oriented on the “upper” sides of the other elements. The exemplary term “lower” can, therefore, encompass both an orientation of lower and upper, depending on the particular orientation of the figure. Similarly, for the terms “horizontal”, “oblique” or “vertical”, in the absence of other clearly defined references, these terms are all relative to the ground. Similarly, if the device in one of the figures is turned over, elements described as “below” or “beneath” other elements will then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present disclosure belongs. It is further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially,” “generally” or “approximately” shall generally mean within 20 percent, preferably within 10 percent, and more preferably within 5 percent of a given value or range. Numerical quantities given herein are approximate, meaning that the terms “around,” “about,” “substantially,” “generally” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the present disclosure.

Embodiments of the present disclosure are illustrated in detail hereinafter with reference to accompanying drawings. It should be understood that specific embodiments described herein are merely intended to explain the present disclosure, but not intended to limit the present disclosure.

In order to further elaborate the technical means adopted by the present disclosure and its effect, the technical scheme of the present disclosure is further illustrated in connection with the drawings and through specific mode of execution, but the present disclosure is not limited to the scope of the implementation examples.

The present disclosure relates to the field of optometry, and more particularly relates to a system for performing an automated refraction examination.

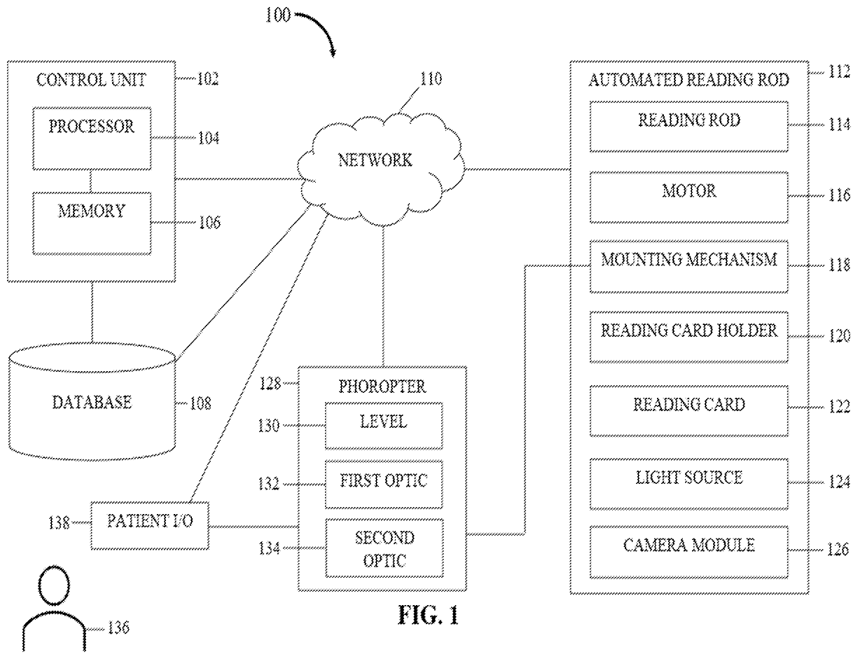

FIG. 1 illustrates a block diagram of an exemplary system for conducting an automated refraction examination, in accordance with an embodiment of the present disclosure.

System 100 may be used to conduct an automated refraction examination with or without an operator for phoropter 128, wherein the operator may be a doctor, an optometrist, a technician, an assistant, etc. System 100 may include control unit 102, database 108, network 110, automated reading rod 112, phoropter 128, and patient I/O 138.

Control unit 102 may be used to control system 100 and may operate autonomously or with inputs from the operator. Control unit 102 may include processor 104 and memory 106, wherein memory 106 may be a non-transitory storage medium holding program instructions for autonomously conducting an automated refraction examination. Processor 104 may be a microcontroller, microprocessor, embedded processor, central processing unit, graphics processing unit, etc. or combination thereof. Memory 106 may be flash memory, ferroelectric random-access memory (FeRAM), magnetic random-access memory (MRAM), phase-change memory (PCM), resistive random access memory (RRAM), etc. Control unit 102 may communicate with patient 136 via one or more output devices, such as, without limitation, a speaker, a display, etc. and may record input from patient 136 via one or more input devices such as, a microphone, a keyboard, a joystick, a camera, etc.

Network 110 may be a wireless connection between automated reading rod 112, phoropter 128, database 108, and control unit 102. While network 110 may be used as a means of communicating between devices of system 100, other means of communicating between automated reading rod 112, phoropter 128, database 108, and control unit 102 may also be used, including alternative wireless communication protocols such as Bluetooth®, 2G, 3G, 4G, 5G, near field communication (NFC), radio frequency identification (RFID), etc. and wired communication protocols such as a universal serial bus (USB), etc. Network 110 may be an internet connection over Wi-Fi, ethernet connection, etc. or a local network such as a local area network (LAN), wide area network (WAN), etc.

Control unit 102 may communicate with database 108 over a wired connection or over network 110. Database 108 may also communicate directly with phoropter 128 and automated reading rod 112 via network 110. Database 108 may store patient information, such as eyeglass prescription data, previous eyeglass prescription data, auto-refractor data, patient personal information, etc. Additionally, status information may be transmitted to database 108 from phoropter 128 or control unit 102 to determine the status of an ongoing refraction examination, including the status of a near vision test. Status information may be used to monitor an ongoing refraction examination through, for example, without limitation, video, images, audio, etc. Status information may also include an exit status used to notify the relevant staff of the completion of a refraction examination. Exit status may be a successful exit or an unsuccessful exit. A successful exit may indicate that the refraction examination exited without any errors. An unsuccessful exit may indicate that an error occurred during the refraction process. During a near vision test, an unsuccessful exit may be, for example, when patient 136 fails to respond after a response period, if a response from patient 136 is unrecognizable, upon receipt of an error signal, etc.

Automated reading rod 112 may be used to conduct the near vision test, wherein automated reading rod 112 includes reading rod 114, motor 116, mounting interface 118, reading card holder 120, reading card 122, light source 124, and camera module 126. Automated reading rod 112 may communicate with phoropter 128 and control unit 102 via a wired connection or a wireless connection, such as network 110.

Reading rod 114 may be mounted to phoropter 128 via mounting interface 118 and may be used as a means for displaying reading card 122 to patient 136. Reading rod 114 may be composed of any suitable material known in the art, such as, without limitation, aluminum, wood, steel, stainless steel, brass, copper, plastic, etc., and may include a level, an accelerometer, a ruler, etc.

Motor 116 may be coupled to reading rod 114 and, when actuated, may move reading rod 114 between an active position and an inactive position. The active position may be when reading rod is parallel to the ground and displaying reading card 122 in front of patient 136. The inactive position may be where reading rod 114 is perpendicular with the ground and stored in an upright position. Motor 116 any suitable motor known in the art, such as, but not limited to, an AC motor, a DC motor, a direct drive motor, a linear motor, a servo motor, etc.

Mounting interface 118 may be used to mount automated reading rod 112 to phoropter 128, and may include, for example, without limitation, a mounting bracket, a mounting block, a mounting adapter, etc.

Reading card holder 120 may be coupled to reading rod 114 at a set distance from patient 136 undergoing a near vision test to display reading card 122 to patient 136. Reading card 122 may be a physical reading rod composed of, for example, paper, plastic, etc., or a digital display. Reading card 122 may display rows of letters, words, shapes, pictures, etc. to patient 136.

Light source 124 may be mounted to reading rod 114 and may be used to illuminate reading card 122. Light source 124 may be a fluorescent light source, an incandescent light source, a light emitting diode (LED). Additionally, light source 124 may be a combination of light sources.

Camera module 126 may be facing the patient and may be used to record video or images of patient 136 or the pupils of patient 136. For example, camera module 126 may be used to record responses from patient 136 such as directional inputs (i.e. patient 136 may point left, right, etc.), hand signs, etc. Camera module 126 may also be used to record images or videos of the pupils of patient 136 to detect neurological problems, such as, without limitation, glaucoma, brain tumors, etc. Camera module 126 may be used stream video to a remote practitioner to supervise the automated refraction examination.

Phoropter 128 may be used to conduct the automated refraction examination, including the automated near vision examination. Phoropter 128 may include level 130, first optic 132, and second optic 134. Level 130 may be used to ensure that phoropter 128 is level. Level 130 may also be a digital level such as, without limitation, an accelerometer. Phoropter 128 may include various hardware configurations to create different optics for conducting a refraction examination, including first optic 132 corresponding to a first view displayed to patient 136 and second optic 134 corresponding to a second view displayed to patient 136. When conducting a refraction examination, various values may be adjusted and optimized to determine an eyeglass prescription for patient 136. The values may include a spherical value, a cylinder value, an axis value, a prism value, an add value, etc. The spherical value may be used to correct for nearsightedness or farsightedness in a patient. The spherical value is measured in diopters (D) and may indicate a lens power required for correcting the patient's vision. The cylinder value and corresponding axis value may be used to correct astigmatism in a patient. The cylinder value may indicate a lens power of a cylinder lens, and I similarly measured in diopters. A positive value may indicate a correction for nearsighted astigmatism, while a negative value may indicate a correction for farsighted astigmatism. The axis value may be measured in degrees and indicates an angle between two meridians of an astigmatic eye. The prism value may be used to correct double vision (i.e. diplopia). The prism value may include a base direction and a power. First optic 132 and second optic 134 may display a view with an added magnifying power value (add value) used to correct presbyopia and may be used for older patients during the near vision test.

When conducting a refraction examination, initial values may be used as a starting point, and adjustments may be made to the initial values to generate optimal values in an eyeglass prescription for patient 136. The initial values may be extracted from patient information stored in database 108 and communicated to control unit 102. Control unit 102 may send signals over network 110 to phoropter 128 such that first optic 132 and second optic 134 may be adjusted based on the initial signals. Phoropter 128 may be placed in front of patient 136 and adjusted such that first optic 132 corresponding to a first view and second optic 134 corresponding to a second view may be positioned in front of a left eye and a right eye of patient 136, respectively. During an automated refraction examination, patient 136 and system 100 may communicate via patient I/O 138. Patient I/O 138 may include input devices configured to record responses from patient 136, such as, without limitation, a microphone, a keyboard, a joystick, a camera, etc. and output devices configured to broadcast messages to patient 136 from system 100, such as, without limitation, a display, a speaker, etc. Patient I/O 138 may be incorporated into phoropter 128 or control unit 102 or may be external devices communicating with phoropter 128 or control unit 102 via network 110. Prior to or during the set up of the automated refraction examination, patient I/O 138 may be initialized according to trial questions and answers communicated to and from patient 136. For example, if using voice recognition, patient 138 may be instructed to read a series of letters or words and the patient's response may be recorded. Patient I/O 138 may calibrate according to the responses recorded from patient 136.

When conducting a refraction examination, a first eye may be tested and corresponding values may be adjusted to determine a right eye prescription. The right eye prescription may include a right sphere value, a right cylinder value, a right axis value, and a right prism value. For each of the right eye values, the patient may be presented two views corresponding to different hardware configurations of phoropter 128 through a left aperture and asked to compare the two views. Control unit 102 may send signals to patient I/O 138 instructing patient I/O 138 to broadcast messages to the patient, including messages asking patient 136 to compare different views. In response, patient 136 my reply using patient I/O 138 and the patient's responses may be communicated to control unit 102. Control unit 102 may calculate updated right eye values and send signals to phoropter 128 over network 110 causing phoropter 128 to adjust the first view and the second view according to the updated right eye values. For example, without limitation, patient 136 may initially be shown a first view and a second view and asked to compare the first view and the second view, wherein the first view may correspond to a first cylinder value and the second view may correspond to a second cylinder value with a corresponding axis offset by 90 degrees. If patient 136 indicates that the second view is clearer via patient I/O 138, control unit 102 may adjust phoropter 128 to show patient 136 a third view and a fourth view, wherein the third view and the fourth view may have increased cylinder values. The comparison process may continue until an optimal right cylinder value is generated, and the optimal right cylinder value may be output to database 108.

Additionally, one view may be shown to patient 136 where the patient is shown a line of letters, words, or images and asked to read the line of letters, words, or images. Based on the patient's performance, adjustments may be made to phoropter 128, continuing until an optimal value is generated. For example, without limitation, phoropter 128 may be initialized according to an initial right eye sphere value and patient 136 may be presented with a line of letters, words, or images via patient I/O 138. Patient 136 may be asked to read the line of letters, words, or images. Patient I/O may record a response from patient 136 via patient I/O 138, and the response may be sent via network 110 to control unit 102. Control unit 102 may adjust the right eye sphere value based on the performance of patient 136, and a new line of letters, words, or images may be presented to patient 136. The process may continue until an optimal right spherical value is generated, and the optimal right sphere value may be output to database 108.

Automated reading rod 112 may be used to conduct an automated near vision test as part of a refraction examination or as an external process separate from the refraction examination. The automated near vision test may be performed on older patients to correct for presbyopia. A determination may be made by control unit 102 to perform the automated near vision test on patient 136 based on patient information stored on database 108. The determination may be based on the age of patient 136, prior prescription data for patient 136, etc. After control unit 102 determines that the automated near vision test is to be conducted, phoropter 128 may be positioned in front of patient 136. Control unit 102 may send a signal to motor 116 such that motor 116 actuates and causes reading rod 114 to from an inactive position into an active position. While in the active position, reading rod 114 may be positioned such that reading card 122 is displayed a short distance from patient 136. In the present embodiment, reading card 122 may be displayed 14 inches from patient 136. However, different distances may also be used and are considered within the scope of the present disclosure. If needed, light source 124 may illuminate reading card 122 to give patient 136 ample lighting to read reading card 122. Light source 124 may be controlled by signals from control unit 102, and/or by an input device of patient I/O 138.

Optionally, camera module 126 may be used to record videos or images of the pupils of patient 136 to determine if patient 136 has a neurological disorder, such as glaucoma or a brain tumor. Camera module 126 may be coupled to reading rod 114 and secured via, for example, without limitation, a screw, a pin, a bolt, adhesive, etc. Camera module 126 may also include one or more secondary light sources for illuminating the eyes of patient 136. Camera module 126 may include a single camera or multiple cameras.

Reference data may be stored on database 108 and accessed by control unit 102 during the automated near vision test. Reference data may include add values corresponding to different patient age ranges. For example, without limitation, a first add value may correspond to patients between the ages of 40 and 45, a second add value may correspond to patients between the ages of 45 and 50, etc. After an add value is determined, control unit 102 may communicate over network 110 such that the spherical hardware settings of phoropter 128 are set to the add value for both eyes of patient 136. Reading card 122 may be a physical reading card containing several lines of letters, words, images, etc. to be displayed to patient 136. The lines may be of incremental sizes, and the letters, words, images, etc. may be different between each line. As such, control unit 102 may determine which line patient 136 is reading based on the content of the line. Alternatively, reading card 122 may be an electronic display controlled by control unit 102, and multiple lines or a single line of letters, words, images, etc. may be shown to the patient. The patient may be instructed to read the smallest clear line of reading card 122. The response of the patient may be recorded via patient I/O 138 and transmitted to control unit 102 over network 110. Control unit 102 may calculate a correction rate of the response of patient 136 by comparing the response to information stored on database 108. The correction rate may be a percentage value, where a higher correction rate indicates more correct responses and a lower correction rate indicates fewer correct responses. If the correction rate is less than a threshold value (e.g. 50%), patient 136 may be instructed to read a next largest line of reading card 122, and a response from patient 136 may be recorded again, repeating the process. If the correction rate is greater or equal to the threshold value, the smallest line size patient 136 was able to successfully read may be recorded by control unit 102 and output to database 108. Control unit 102 may then detect that the automated near vision test is complete and sends a signal to automated reading rod 112, turning off light source 125 and camera module 126 and causing motor 116 to actuate. As such, reading rod 114 may be placed in the inactive position and securely stowed away.

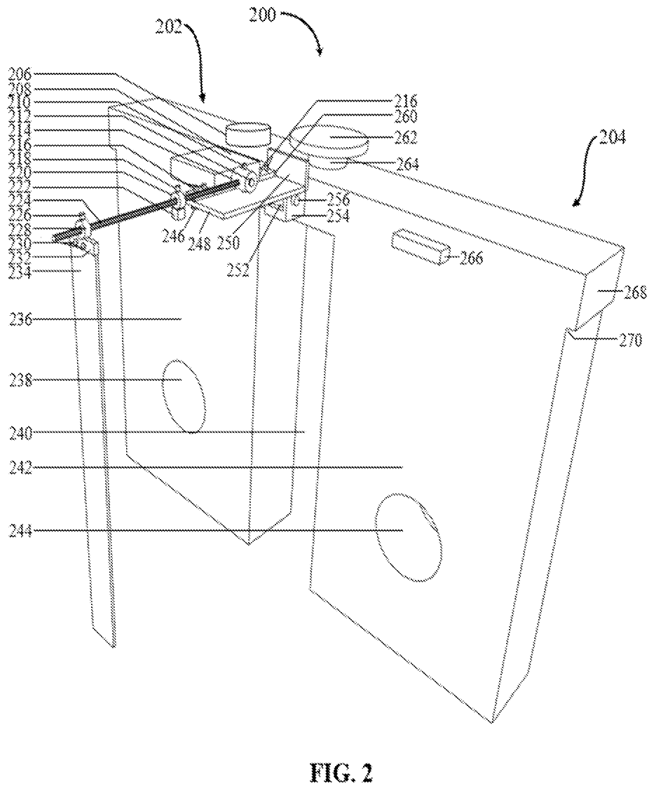

FIG. 2 illustrates an exemplary automated refraction system, in accordance with an embodiment of the present disclosure. Automated refraction system 200 may be used to conduct an automated refraction without the use of an operator. A near vision reading test may be used as part of an automated refraction examination to determine a patient's visual acuity while viewing a vision target at a close proximity.

Automated refraction system 200 includes automated reading rod 202, phoropter 204, and control unit 206, wherein automated reading rod 202 is mounted to phoropter 204 via first mounting adapter 254. In one embodiment, phoropter 204 includes automated reading rod 202 and control unit 206.

Automated reading rod 202 includes motor 208, motor shaft pin 210, motor shaft 212, attachment ring 214, motor mounting pins 216, light source pin 218, light source mount 220, light source 222, reading rod 224, reading card pin 226, reading card mount 228, reading card holder pins 230, reading card holder 232, reading card 234, motor mounting nuts 246, mounting bracket 248, backplate 250, mounting block 252, first mounting adapter 254, mounting block pin 256, and motor mounting plate 260.

Phoropter 204 includes right member 236, right aperture 238, middle gap 240, left member 242, left aperture 244, phoropter mount 262, phoropter mount base 264, level 266, upper member 268, and lower portion 270.

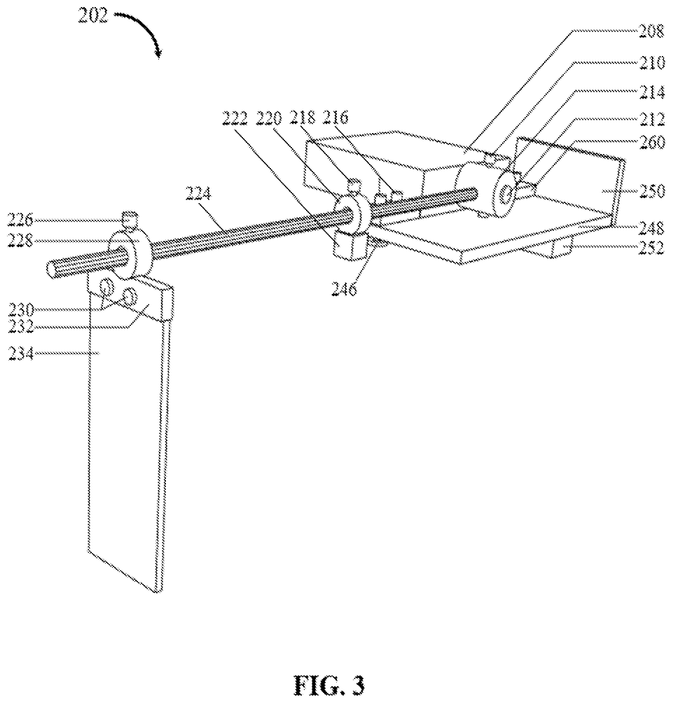

Automated reading rod 202 and phoropter 204 will be described in greater detail below with reference to FIGS. 3-5 and 6A-6B.

Control unit 206 may be internal or external to phoropter 204. Control unit 206 may be used to control automated refraction system 200 and may communicate with the components of automated refraction system 200 via a wired or wireless connection. Control unit 206 may be, for example, without limitation, a computer, a mobile device, a tablet, a microcontroller, etc.

FIG. 3 illustrates an exemplary automated reading rod, in accordance with an embodiment of the present disclosure. With reference to both FIG. 2 and FIG. 3, automated reading rod 202 may be used to display reading card 234 at an appropriate distance in front of a patient conducting a near vision test. Automated reading rod 202 includes motor 208, motor shaft pin 210, motor shaft 212, attachment ring 214, motor mounting pins 216, light source pin 218, light source mount 220, light source 222, reading rod 224, reading card pin 226, reading card mount 228, reading card holder pins 230, reading card holder 232, reading card 234, motor mounting nuts 246, mounting bracket 248, backplate 250, mounting block 252, first mounting adapter 254, mounting block pin 256, and motor mounting plate 260.

Reading rod 224 may be used to display reading card 234 at a set distance in front of the patient. While in the present embodiment, reading rod 224 is a cylindrical shape with a circular cross section, as will be appreciated by one skilled in the art, reading rod 224 may be of any appropriate shape and cross section, such as, without limitation, rectangular, hexagonal, ovular, triangular, etc. Reading rod 224, as shown in FIG. 3 is an exemplary reading rod used as part of automated reading rod 202. Reading rod 224 may have different dimensions than shown, wherein the dimensions may include length, width, diameter, etc. Reading rod 224 may be coupled to motor shaft 212 of motor 208 via motor shaft pin 210. Additionally, reading rod 224 may be positioned in an active position or an inactive position. As shown, reading rod 224 is in the active position, parallel with the ground, so as to display reading card 234 to the patient during an automated near vision test. When not in use, reading rod 224 may be placed in a vertical position by rotation about motor shaft 212 of motor 208 such that reading rod 224 is perpendicular with the ground. When placed in the inactive position, reading rod 224 may be stowed away such that phoropter 204 (not shown) may be used for the automated refraction examination. Reading rod 224 may also include a ruler along its length such that reading card holder 232 may be positioned at a precise distance away from the patient. In one embodiment, the distance of reading card holder 232 may be automatically adjusted using motor 208. In the present embodiment, reading rod 224 is composed of aluminum. However, reading rod 224 may be alternatively composed of, for example, without limitation, copper, brass, stainless steel, wood, plastic, etc.

Motor 208 may be used to drive reading rod 224 between the active position and the inactive position. Motor 208 may include motor shaft 212 coupled to attachment ring 214 of reading rod 224. In the present embodiment, attachment ring 214 is coupled to motor shaft 212 via motor shaft pin 210. However, alternative coupling means between motor shaft 212 and attachment ring 214 may also be used, such as, without limitation, adhesive, welding, screws, fasteners, rivets, etc. Motor 208 any suitable motor known in the art, such as, but not limited to, an alternating current (AC) motor, a during current (DC) motor, a direct drive motor, a linear motor, a servo motor, etc.

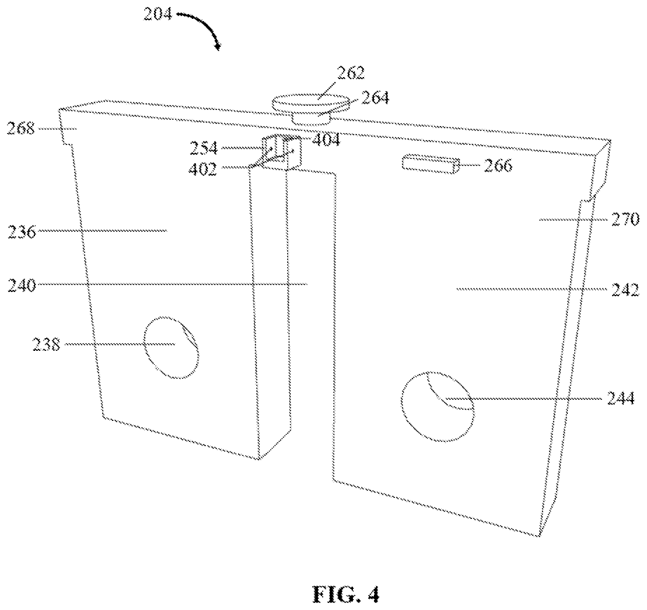

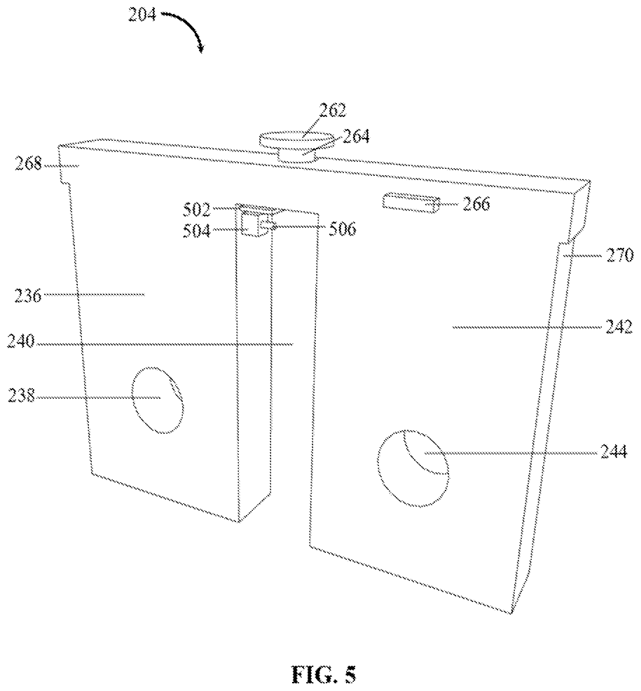

In the present embodiment, the mounting interface of automated reading rod 202 may include mounting bracket 248, backplate 250, mounting block 252, and first mounting adapter 254 (to be described below with reference to FIGS. 4-5). Mounting bracket 248 and backplate 250 may be used to support the other components of automated reading rod 202, especially motor 208. Motor 208 may be coupled to mounting bracket 248 via motor mounting pins 216, motor mounting nuts 246 and motor mounting plate 260. Mounting bracket 248 may be coupled to mounting block 252 via an adhesive. However, as will be appreciated by one skilled in the art, motor 208 may be coupled to mounting bracket 248, mounting bracket 248 may be coupled to backplate 250, and likewise mounting bracket 248 may be coupled to mounting block 252 by a variety of different means, such as, without limitation, rivets, adhesive, fasteners, bolts, screws, etc. Mounting block 252 may be coupled to first mounting adapter 254 via mounting block pin 256. Alternative mounting means may also be used to couple mounting adapter 254 to mounting block 252, such as, without limitation, adhesive, fasteners, bolts, screws, etc.

Automated reading rod 202 may be mounted to phoropter 204 via a variety of different means. As disclosed herein, phoropter 204 may be mounted at different locations, as will be described in FIGS. 4-5, and using different mounting adapters. However, as will be appreciated by one skilled in the art, the mounting interfaces for automated reading rod 202 are not limited to the embodiments described herein, and alternative mounting means may be used to mount automated reading rod 202 to phoropter 204. For example, without limitation, a subset of the aforementioned mounting interface may be used, additional elements may be incorporated, different configurations may be applied, etc.

Light source 222 may be used to illuminate reading card 234 when in use and may be mounted to reading rod 224 via light source mount 220 and light source pin 218. Light source pin 218 may be threaded into light source mount 220 to securely couple light source 222 to reading rod 224. The tip of light source pin 218 may be a soft material, such as, without limitation, silicone, rubber, latex, etc. such that, upon threading light source pin 218 into the threads of light source mount 220, the tip of light source pin 218 may contact reading rod 224 and compress the soft material. The tension between the soft material and reading rod 224 may keep light source 222 in place. Light source 222 may be positioned about the length of reading rod 224 and may be angled such that a portion of or the entirety of reading card 234 is illuminated. Light source 222 may be, for example, without limitation, an LED light source, an incandescent light source, a fluorescent light source, etc. Additionally, light source 222 may comprise one or more individual light sources, wherein the individual light sources may be a combination of incandescent light sources, fluorescent light sources, LED light sources, etc. or all individual light sources may be the same type of light source.

Reading card holder 232 and reading card holder pins 230 may be utilized to secure reading card 234 to reading rod 224 at an appropriate distance from the patient. Reading card holder 232 may be secured to reading rod 224 via reading card mount 228 and reading card pin 226. Similar to light source mount 220 and light source pin 218, reading card pin 226 may have a tip composed of a soft material such as, without limitation, silicone, rubber, latex, etc., and may be threaded into reading card mount 228 to secure reading card holder 232 to reading rod 224 using the soft material of the tip of reading card pin 226. Reading card 234 may be of a variety of different shapes and sizes and is not limited to the configuration shown in FIG. 3. For example, reading card may be triangular, circular, square-shaped, a complex shape, etc. Additionally, reading card 234 may be physical or digital. For example, without limitation, reading card 234 may be composed of paper, plastic, aluminum, etc. with printed lines of letters, words, images, etc. in a physical embodiment, or may utilize a screen or projector in a digital embodiment to display one or more digitally presented lines of letters, words, images, etc. In the physical embodiment, the thickness of reading card 234 in the present embodiment may be 0.1 mm. However, reading card 234 may be of different thicknesses depending on the material used and the specific utilization.

FIG. 4 illustrates an exemplary phoropter with a first mounting adapter, in accordance with an embodiment of the present disclosure. With reference to FIG. 2 and FIG. 4, phoropter 204 includes right member 236, right aperture 238, middle gap 240, left member 242, left aperture 244, phoropter mount 262, phoropter mount base 264, level 266, upper member 268, and lower portion 270.

Phoropter 204 may be a manual or digital phoropter including right aperture 238 corresponding to a patient's right eye and left aperture 244 corresponding to the patient's left eye. The patient may be shown a first view and a second view through one or more of a first optic and a second optic of right aperture 238 and left aperture 244, respectively. The first and second optics may be produced using a variety of different lenses. In one embodiment, liquid crystal lenses may be used to produce the first and second optics. Phoropter 204 may be used when conducting the automatic refraction examination.

Upper member 268 of phoropter 204 may be the raised portion of phoropter 204 connecting right member 236 and left member 242. Left member 242 and right member 236 may be components of lower portion 270. Upper member 268 may include mounting locations for first mounting adapter 254, level 266, etc. Upper member 268 may be mounted to, for example, without limitation, an ophthalmic stand during an automated refraction examination.

In the present embodiment, first mounting adapter 254 may be mounted onto a front face of upper member 268 between right member 236 and left member 242 and above middle gap 240 such that first mounting adapter 254 is equidistant from right aperture 238 and left aperture 244. First mounting adapter 254 may be mounted to the front face of upper member 268 via back mounting adapter screw 404. Alternatively, first mounting adapter 254 may be mounted anywhere along upper member 268, on right member 236, or on left member 242. First mounting adapter 254 may be coupled to mounting block 252 (not shown) of automated reading rod 202 or a mounting block of a manual reading rod via first mounting block holes 402.

Phoropter 204 may be, for example, without limitation, wall mounted, mounted using an ophthalmic stand, etc. via phoropter mount 262 and phoropter mount base 264. Phoropter mount 262 may be coupled to an ophthalmic stand or other suitable mounting medium, and the diameter of phoropter mount 262 may vary depending on the type of mounting medium. Phoropter mount base 264 may be coupled to phoropter mount 262 and upper member 268.

Phoropter 204 may also include level 266 for adjusting the 3D positioning of phoropter 204 and ensuring that phoropter 204 is parallel with the ground. In one embodiment, level 266 may be an accelerometer. In another embodiment, level 266 may be mounted onto automated reading rod 202.

FIG. 5 illustrates an exemplary phoropter with a second mounting adapter, in accordance with an embodiment of the present disclosure. As described with reference to FIG. 4, phoropter 204 includes right member 236, right aperture 238, middle gap 240, left member 242, left aperture 244, phoropter mount 262, phoropter mount base 264, level 266, upper member 268, and lower portion 270. Second mounting adapter 502 may be mounted at a different location than first mounting adapter 254. In the present embodiment, second mounting adapter 502 may be mounted at a lower surface of upper member 268 between left member 242 and right member 236 of phoropter 204. As such, motor 504 may be more directly incorporated with phoropter 204. Second mounting adapter 502 may be mounted to motor 504 with motor shaft 506. Motor 504 may be the same motor 208 described in FIG. 2 adapted to be mounted using second mounting adapter 502. Second mounting adapter 502 may be mounted using a variety of different means, such as, without limitation, screws, bolts, adhesive, etc.

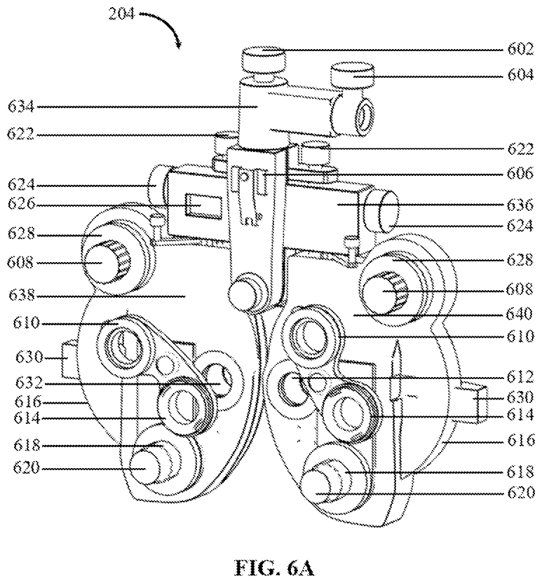

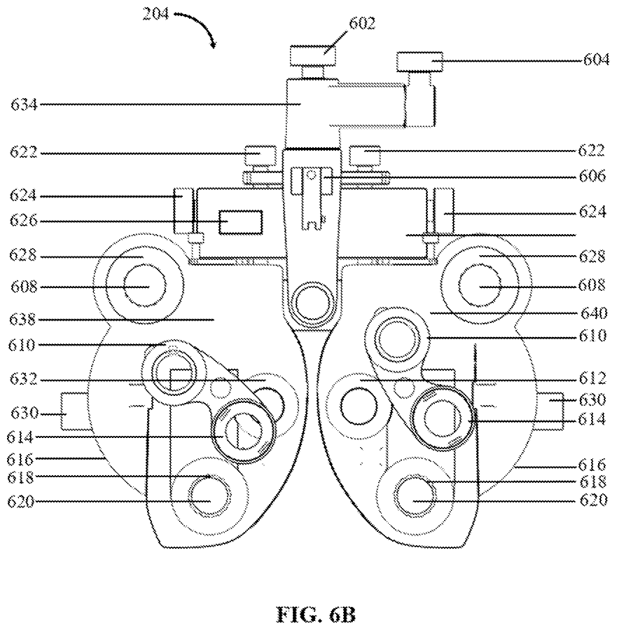

FIGS. 6A-6B illustrate an exemplary phoropter, wherein FIG. 6A shows a perspective view of a phoropter and FIG. 6B shows a front view of a phoropter, in accordance with an embodiment of the present disclosure.

Phoropter 204 may include rotation adjustment knob 602, tilt clamp knob 604, third mounting adapter 606, auxiliary lens knobs 608, Jackson Cross Cylinder (JCC) units 610, left aperture 612, prism units 614, weak sphere control dials 616, cylinder axis knobs 618, cylinder power knobs 620, leveling knobs 622, pupillary distance (PD) knobs 624, PD scale 626, strong sphere control knobs 628, vertex distance alignment units 630, right aperture 632, upper mount 634, main body 636, right member 638, and left member 640.

One skilled in the art may understand the interconnections of the components of phoropter 204.

Tilt clamp knob 604 may be used to mount phoropter 204 to an ophthalmic stand (not shown) and may be used to release phoropter 204 such that phoropter 204 may be stored separate from the ophthalmic stand. Tilt clamp knob 604 may also be used to adjust the tilt of phoropter 204. Tilt clamp knob 604 may be coupled to a first end of upper mount 634.

Rotation adjustment knob 602 may be used to adjust the rotation of main body 636 of phoropter 204 such that phoropter 204 may be positioned directly in front of the patient or such that phoropter 204 may be stored away. Rotation adjustment knob 602 may be coupled to a second end of upper mount 634.

Upper mount 634 may be coupled to main body 636 via any means known in the art. Main body 636 may similarly be mounted to right member 638 and left member 640. Right member 638 may be positioned such that right aperture 632 is placed in front of the patient's right eye while left member 640 may be positioned such that left aperture 612 is placed in front of the patient's left eye.

Third mounting adapter 606 may be used to mount automated reading rod 202 to phoropter 204 and may be coupled to main body 636. Many types of mounting adapters may be used when mounting automated reading rod 202, and third mounting adapter 606 may be secured to one of several mounting locations about phoropter 204. Additionally, a curved or bent reading rod may be coupled to third mounting adapter 606 such that automated reading rod 202 may be mounted at an off center mounting location. Mounting at an off center mounting location may free space on main body 636 of phoropter 204.

As will be appreciated by one skilled in the art, while a first phoropter component of phoropter 204 may be used to adjust a left optic of left aperture 612 when testing a patient's left eye, an identical, mirrored component of phoropter 204 may be used to adjust a second optic of left aperture 612 of the patient's right eye.

Auxiliary lens knobs 608 may be used to occlude or open left aperture 612 and right aperture 632, as well as switch between different lenses to conduct various visual acuity tests. Auxiliary lens knobs 608 may be coupled to left member 640 and right member 638 of phoropter 204 as auxiliary lens knobs 608 are used to adjust the left optic of left aperture 612 for the patient's left eye and the right optic of right aperture 632.

Jackson Cross Cylinder (JCC) units 610 may be used during a refraction examination in adjusting a cylinder value and a corresponding axis value to correct for astigmatism of the patient. JCC units 610 may share a housings with prism units 614, and the housings may be coupled to left member 640 and right member 638 such that JCC units 610 or prism units 614 may be placed in front of the patient's left and right eyes when testing the patient's cylinder and prism values, respectively.

Left aperture 612 may be placed in front of the patient's left eye and when in use, different optics may be used to display different views to the patient. As the patient compares different views, updates to the patient's eyeglass prescription values (i.e. spherical value, cylinder value, axis value, etc.) may be made to optimize the patient's visual acuity. Left aperture 612 may be a hollow opening of left member 640 through which the patient may view different visual targets through different lenses using the patient's left eye.

Prism units 614 may be used when adjusting prism values of a patient. The prism values may be used to correct diplopia (i.e. double vision) and may share the same housings as JCC units 610.

Weak sphere control dials 616 may be used to finely tune spherical values shown to the patient. In the present embodiment, weak sphere control dials 616 may have a step of 0.25 diopters. However, different step sizes may also be used and are within the scope of the current embodiment. Weak sphere control dials 616 may be coupled to left member 640 and right member 638 of phoropter 204 and used to adjust the spherical values of the right optic and the left optic.

Cylinder axis knobs 618 may be used to adjust the axis of cylindrical lenses when correcting for astigmatism. Cylinder axis knobs 618 may be coupled to left member 640 and right member 638 of phoropter 204 and used to adjust the cylinder axis values of the right optic and the left optic.

Cylinder power knobs 620 may be used to adjust a cylindrical lens power when correcting for astigmatism. Cylinder power knobs 620 may be coupled to cylinder axis knobs 618 such that cylinder lens power and cylinder axis may be easily controlled for the patient's left and right eyes.

Leveling knobs 622 may be used to adjust the level position of phoropter 204 such that phoropter 204 may be placed in an optimal position when conducting a refraction examination. Leveling knobs 622 may be coupled to main body 636 of phoropter 204, where leveling knobs 622 may separately control the level of right member 638 and left member 640.

PD knobs 624 may be used to adjust the pupillary distance of phoropter 204 to match up with the pupillary distance of the patient, wherein pupillary distance is the distance between the centers of the patient's pupils. Pupillary distance knob may be coupled to main body 636, and may adjust the pupillary distance for the patient's right eye and left eye separately or together.

PD scale 626 may be used to display the current pupillary distance value of phoropter 204. PD scale 626 may be coupled to main body 636 and may communicate via a wired or wireless connection with PD knobs 624 to show the current pupillary distance value. PD scale 626 may show a pupillary distance of the right and the left eyes of the patient.

Strong sphere control knobs 628 may be used to make large adjustments to the spherical value shown to the patient. In the present embodiment, strong sphere control knobs 628 may be used to adjust the spherical value with a step of 3.00 diopters. However, different step sizes may also be used and are considered within the scope of the present embodiment. Strong sphere control knobs 628 may be coupled to right member 638 and left member 640 of phoropter 204 and may be used to adjust the sphere value for the patient's right and left eyes.

Vertex distance alignment units 630 may be used to display the position of the patient's cornea vertexes. Vertex distance may be the distance between the back surface of the lens of the phoropter and the front surface of the patient's eye. Vertex distance alignment units 630 may be used to view the vertex distance of the patient's right and left eyes. Vertex distance alignment units 630 may be coupled to right member 638 and left member 640 of phoropter 204.

Right aperture 632 may be placed in front of the patient's right eye and when in use, different optics may be used to display different views to the patient. As the patient compares different views, updates to the patient's eyeglass prescription values (i.e. spherical value, cylinder value, axis value, etc.) may be made to optimize the patient's visual acuity. Right aperture 632 may be a hollow opening of right member 638 through which the patient may view different visual targets through different lenses using the patient's right eye.

While automated reading rod 202 may be mounted to phoropter 204, automated reading rod 202 may also be adapted to be mounted on conventional phoropters, such as digital phoropters, manual phoropters, etc.

To use phoropter 204 for an automated near vision test, if stored away, tilt clamp knob 604 may be used to attach upper mount 634 to an ophthalmic stand or other similar stand. Phoropter may be positioned in front of the patient via rotation adjustment knob 602 and leveling knobs 622. An appropriate pupillary distance may be determined (i.e. beforehand or during the automated near vision test) and phoropter 204 may be adjusted to the determined pupillary distance via PD knobs 624. Vertex distance alignment units 630 may also be used to measure and adjust the distance between the eyes of the patient and the lenses in left aperture 612 and right aperture 632.

An external database or reference chart may be used to determine an add value for the patient. Automated reading rod 202 may be attached to phoropter 204 via third mounting adapter 606. Alternatively, other mounting interfaces may be used to attach automated reading rod 202, such as the first mounting adapter, the second mounting adapter, etc. Motor 208 of automated reading rod 202 may be actuated resulting in reading rod 224 of automated reading rod 202 to be placed into an active position, with a reading card in front of the patient. The reading card may display lines of letters, words, images, etc., wherein the lines may be letters, words, images, etc. of different sizes. The patient may view the reading card through left aperture 612 and right aperture 632. The spherical power of the lenses in left aperture 612 and right aperture 632 may be adjusted according to the determined add value via strong sphere control knobs 628 and weak sphere control dials 616. The patient may be instructed to read the smallest clear line of the lines of letters, words, images, etc. Data may be gathered based on the patient's response, and the patient may be instructed to read a line with larger sized letters, words, images, etc. The data may be output to a database, and upon completion of the automated near vision test, motor 208 may be actuated such that reading rod 224 may be placed in an inactive position.

The components of phoropter 204 may also be adjusted digitally. For example, control 206 unit may communicate with phoropter 204 such that control unit 206 may automatically select lenses to create different optics when performing an automated refraction examination. Phoropter 204 and control unit 206 may also communicate with the patient via patient I/O devices, such as, without limitation, speakers, cameras, microphones, displays, joysticks, buttons, keyboards, lights, etc.

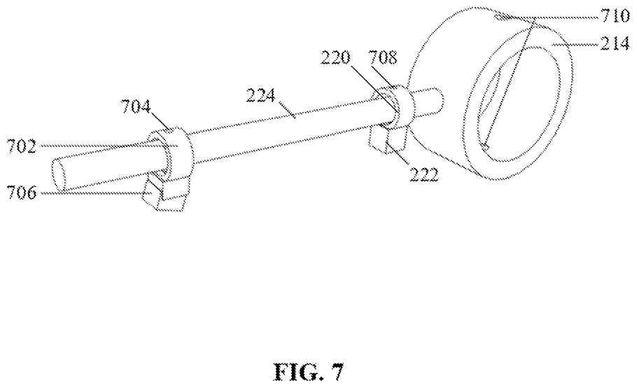

FIG. 7 illustrates an exemplary camera module, in accordance with an embodiment of the present disclosure. With reference to both FIG. 3 and FIG. 7, camera module 706 may be used in an automated refraction system to analyze a pupil response of the patient. Camera module 706 may be secured to reading rod 224 such that camera module 706 is positioned between the patient's eyes. Reading rod 224 may be mounted to motor shaft 212 (not shown) via attachment ring 214 and motor shaft pin holes 710. Additional elements may be simultaneously secured to reading rod 224, such as light source 222, reading card holder 232 (not shown) etc. Light source 222 may be secured to reading rod 224 via light source mount 220 and light source screw hole 708. The additional elements and camera module 706 may be positioned at any point along the length of reading rod 224. For example, without limitation, the positions of camera module 706 and light source 222 may be swapped.

In the present embodiment, camera module 706 may be mounted to reading rod 224 via camera module mount 702 and mounting hole 704. For example, without limitation, a screw with a soft tip may be inserted into mounting hole 704, thus securing camera module 706 to reading rod 224. The soft tip may be composed of, for example, silicone, rubber, etc. Other mounting means may also be used to secure camera module 706 to reading rod 224, such as, without limitation, rivets, pins, screws, bolts, adhesive, clamps, etc.

Camera module 706 may house various components used to observe a pupil response of the patient. In the present embodiment, camera module 706 includes a left light source for illuminating the patient's left eye, a right light source for illuminating the patient's right eye, and a camera positioned between the left light source and the right light source. The left light source and the right light source may be, for example, without limitation, LEDs, incandescent light sources, fluorescent light sources, etc. Alternatively, only one light source may be used to illuminate both eyes, or a greater number of light sources may be used. The camera may be used to record images or video of the client's right and left eyes. In one embodiment, multiple cameras may be incorporated within camera module 706. For example, a first camera may be used to record the pupil response of the patient's right eye, while a second camera may be used to record the pupil response of the patient's left eye. The camera may record images or video of the patient's eyes simultaneously or sequentially. For example, the right light source may illuminate the patient's right eye and the camera may record images or video of the right eye. Subsequently, the left light source may illuminate the patient's left eye and the camera may record images or video of the left eye. Additionally, the camera may record both eyes simultaneously while only one light source is illuminated. The video or images of the patient's eyes may be used to determine if the patient has a neurological problem, such as, but not limited to, glaucoma, brain tumors, etc.

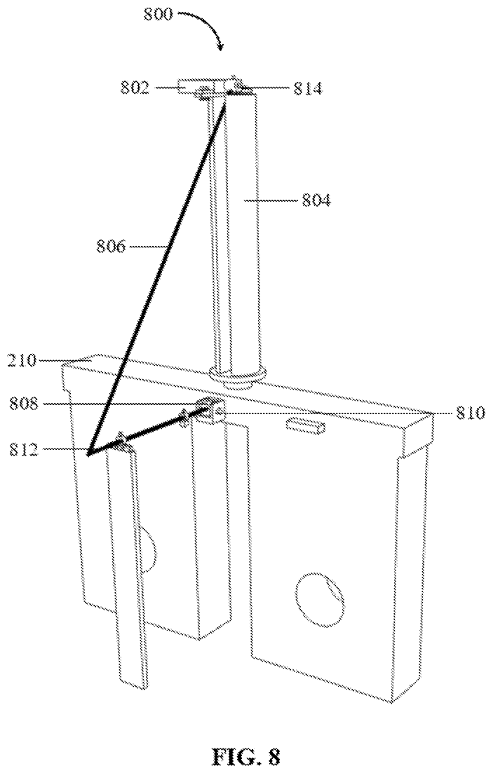

FIG. 8 illustrates an automated refraction cable system, in accordance with an embodiment of the present disclosure. Automated refraction cable system 800 may include motor 802, mount 804, cable 806, reading rod 812, mounting block 808, and mounting adapter 810.

Motor 802 may be any suitable motor known in the art, such as, but not limited to, an AC motor, a DC motor, a direct drive motor, a linear motor, a servo motor, etc. Motor 802 may be used to transition reading rod 812 between an active position and an inactive position via cable 806. If reading rod 812 is in the active position (as shown in FIG. 8), upon actuation of motor 802, cable 806 may spool about motor shaft 814, pulling reading rod 812 into an inactive position. Additionally, a spool may be attached to motor shaft 814 such that cable 806 may be securely stored.

Mount 804 may be used to position motor 802 at an appropriate position to allow for motor 802 to efficiently raise or lower reading rod 812. Mount 804 as depicted is cylindrical in shape. However, as will be appreciated by one skilled in the art, the shape and size of mount 804 may vary depending on the specific application. For example, without limitation, mount 804 may be rectangular, hexagonal, triangular, etc. Further, properties of mount 804 such as diameter, height, etc. may also vary. Mount 804 may be composed of any suitable material, such as, but not limited to, steel, aluminum, copper, brass, wood, plastic, etc.

Cable 806 may be flexible enough to coil about motor shaft 814, yet strong enough to support the weight of reading rod 812. Cable 806 may be, for example, without limitation, rope, wire, etc. Cable 806 may be secured to reading rod 812 via, for example, without limitation, a knot, adhesive, fasteners, a loop, etc.

Mounting block 808 and mounting adapter 810 may be one means of mounting reading rod 812 to phoropter 204. Various alternative mounting means may also be used, so long as ample clearance is provided to allow for reading rod 812 to be moved between an active position and an inactive position.

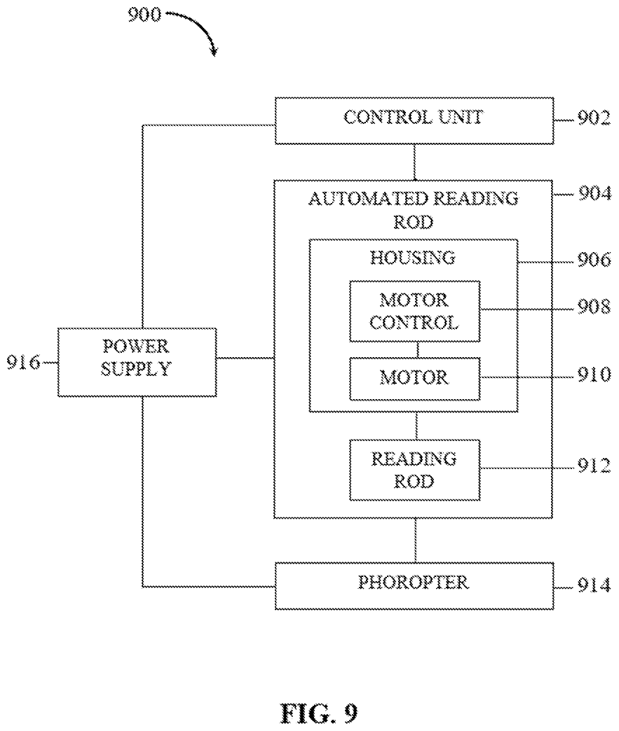

FIG. 9 illustrates an exemplary optometry examination system, in accordance with an embodiment of the present disclosure. Optometry examination system 900 may include control unit 902, automated reading rod 904, phoropter 914, and power supply 916. Control unit 902, automated reading rod 904, and phoropter 914 may be connected via a wireless or wired connection, while power supply may be connected to control unit 902, automated reading rod 904, and phoropter 914 via a wired connection.

Control unit 902 may be a computer, microcontroller, mobile device, tablet, etc. used to communicate with and control automated reading rod 904 and phoropter 914. Control unit 902 may send signals to automated reading rod 904 and phoropter 914 during a refraction examination.

Automated reading rod 904 may include housing 906 and reading rod 912, wherein housing 906 may be to protect the components of a motor mechanism.

The motor mechanism, including motor control 908 and motor 910, may be coupled to reading rod 912 such that the motor mechanism may be used to switch reading rod 912 between an active position and an inactive position. Housing 906 may be used to protect motor control 908 and motor 910, and may be composed of, for example, without limitation, plastic, aluminum, etc.

Motor control 908 may communicate with control unit 902 to actuate motor 910. Control unit 902 may send signals to motor control 908, and motor control 908 may actuate to control reading rod 912. Alternatively, motor control 908 may communicate directly with phoropter 914.

Power supply 916 may be one power source used to power control unit 902, automated reading rod 904, and phoropter 914, or multiple power sources for powering control unit 902, automated reading rod 904, and phoropter 914. In one embodiment, one power source may be used to power control unit 902, while another power source may be used to power automated reading rod 904 and phoropter 914. Power supply 916 may be an unregulated power supply, a linear regulated power supply, a switching power supply, etc. Power supply 916 may also be a battery, renewable energy source, or other energy source known in the art.

The foregoing description of the present disclosure, along with its associated embodiments, has been presented for purposes of illustration only. It is not exhaustive and does not limit the present disclosure to the precise form disclosed. Those skilled in the art will appreciate from the foregoing description that modifications and variations are possible considering the said teachings or may be acquired from practicing the disclosed embodiments.

Likewise, the steps described need not be performed in the same sequence discussed or with the same degree of separation. Various steps may be omitted, repeated, combined, or divided, as necessary to achieve the same or similar objectives or enhancements. Accordingly, the present disclosure is not limited to the said-described embodiments, but instead is defined by the appended claims considering their full scope of equivalents.

What is claimed is:

1. A phoropter for conducting an automated refraction examination on a patient comprising:
a right member with a right aperture;
a left member with a left aperture;
an upper member connecting to the right member and the left member;
a mounting interface coupled to the upper member;
a control unit configured to send signals to conduct the automated refraction examination; and
an automated reading rod mounted to the phoropter via the mounting interface, the automated reading rod comprising:
a reading rod comprising a first end and a second end;
a motor coupled to the first end of the reading rod, wherein the motor is configured to drive the reading rod between a first position and a second position;
a reading card holder coupled to the second end of the reading rod; and
a reading card coupled to the reading card holder.

2. The phoropter of claim 1, wherein the control unit is configured to communicate with the patient via an input device and an output device;
wherein the output device comprises one or more of a speaker and a display; and
wherein the input device comprises one or more of a microphone, a keyboard, a joystick, and a camera.

3. The phoropter of claim 2, wherein the control unit communicates with the patient to adjust the phoropter during an automated refraction examination.

4. The phoropter of claim 3, wherein the phoropter is configured to output results of the automated refraction examination to a database.

5. The phoropter of claim 1, wherein the mounting interface comprises:
a mounting adapter coupled to the phoropter;
a mounting block coupled to the mounting adapter; and
a mounting bracket coupled to the mounting block;
wherein the motor is coupled to the mounting bracket.

6. The phoropter of claim 1, wherein the first position is an active position and the second position is an inactive position.

7. The phoropter of claim 1, wherein the motor further comprises a motor shaft and a cable is coupled to the motor shaft and to the second end of the reading rod such that actuating the motor drives the reading rod between the first position and the second position.

8. The phoropter of claim 1, further comprising a light source, wherein the light source is coupled to the reading rod between the reading card holder and the motor and is configured to illuminate the reading card.

9. The phoropter of claim 8, wherein the control unit controls the light source to illuminate the reading card.

10. The phoropter of claim 1, further comprising a camera module, wherein the camera module is coupled to the reading rod between the reading card holder and the motor and is configured to capture images of the patient's pupils.

11. The phoropter of claim 9, wherein the camera module comprises one or more secondary light sources configured to illuminate one or more pupils of the patient.

12. The phoropter of claim 11, wherein the control unit controls the camera module to capture images of the patient's pupils.

13. The phoropter of claim 5, wherein the mounting adapter is coupled to a front surface of the upper member.

14. The phoropter of claim 5, wherein the mounting adapter is coupled to the phoropter between the left member and the right member to a lower surface of the upper member.

15. The phoropter of claim 1, wherein the reading card is an electronic display.

16. The phoropter of claim 15, wherein the control unit controls the electronic display to display lines of letters, words or images to the patient.

17. The phoropter of claim 1, wherein the controller is configured to communicate wirelessly with the motor.

18. The phoropter of claim 1, wherein the controller communicates via a wired connection with the motor.

19. The phoropter of claim 1, wherein the phoropter further comprises a level indicating a balance status of the phoropter.

20. The phoropter of claim 19, wherein the level is a digital level.
